(19) United States
(12) Patent Application Publication
(10) Pub. No.: US 2016/0007935 A1
(43) Pub. Date: Jan. 14, 2016

(54) Methods and apparatus for measuring physiological parameters

(71) Applicants: Javier Hernandez, Cambridge, MA (US); Daniel McDuff, Cambridge, MA (US); Rosalind Picard, Newton, MA (US)

(72) Inventors: Javier Hernandez, Cambridge, MA (US); Daniel McDuff, Cambridge, MA (US); Rosalind Picard, Newton, MA (US)

(73) Assignee: MASSACHUSETTS INSTITUTE OF TECHNOLOGY, Cambridge, MA (US)

(21) Appl. No.: 14/861,388

(22) Filed: Sep. 22, 2015

Related U.S. Application Data
(63) Continuation-in-part of application No. 14/661,747, filed on Mar. 18, 2015.
(60) Provisional application No. 62/053,802, filed on Sep. 23, 2014.
(60) Provisional application No. 62/053,805, filed on Sep. 23, 2014.
(60) Provisional application No. 62/103,782, filed on Jan. 15, 2015.
(60) Provisional application No. 61/955,772, filed on Mar. 19, 2014.

(51) Int. Cl. A61B 5/00; A61B 5/0205; A61B 5/117

(52) U.S. Cl. CPC A61B 5/7278; A61B 5/6814; A61B 5/6824; A61B 5/117; A61B 5/0077; A61B 5/0205; A61B 2562/0219; A61B 5/1118

(57) ABSTRACT

A sensor system includes one or more gyroscopes and one or more accelerometers, for measuring subtle motions of a user's body. The system estimates physiological parameters of a user, such as heart rate, breathing rate and heart rate variability. When making the estimates, different weights are assigned to data from different sensors. For at least one estimate, weight assigned to data from at least one gyroscope is different than weight assigned to data from at least one accelerometer. Also, for at least one estimate, a weight assigned to one or more sensors located in a first region relative to the user's body is different than a weight assigned to one or more sensors located in a second region relative to the user's body. Furthermore, weight assigned to data from at least one sensor changes over time.

### RELATED APPLICATIONS

This application claims the benefit of U.S. Provisional Application No. 62/053,802, filed Sep. 23, 2014 (the “802 Application”), U.S. Provisional Application No. 62/053,805, filed Sep. 23, 2014 (the “805 Application”), and U.S. Provisional Application No. 62/103,782, filed Jan. 15, 2015 (the “782 Application”). This application is a continuation-in-part of U.S. application Ser. No. 14/661,747, filed Mar. 18, 2015 (the “747 Application”), which claims the benefit of U.S. Provisional Application No. 61/955,772, filed Mar. 19, 2014 (the “772 Application”). The entire disclosures of the 802 Application, 805 Application, 782 Application, 747 Application and 772 Application are incorporated herein by reference.

### STATEMENT REGARDING FEDERALLY SPONSORED RESEARCH OR DEVELOPMENT

This invention was made with government support under Grant No. IIS-1029585 awarded by the National Science Foundation. The government has certain rights in the invention.

### FIELD OF TECHNOLOGY

The present invention relates generally to dynamic weighting of data from a set of sensors, such that weighting changes over time and depends on at least (i) type of sensor and (ii) sensor position relative to a user's body.

### SUMMARY

In illustrative implementations, a sensor system includes one or more accelerometers, gyroscopes and optical sensors. For example, in some cases, the optical sensors comprise photoplethysmographic (PPG) sensors. The gyroscopes and accelerometers measure subtle body movements caused by heartbeats and breathing. The PPG sensors measure blood volume pulse and other subtle body changes caused by heartbeats and breathing.

The sensor system (a) takes sensor measurements of a human user; (b) based on the sensor measurements, calculates a cardiac waveform and a respiratory waveform; (c) based on the waveforms, calculates heart rate, breathing rate and other physiological parameters, such as heart rate variability; and (d) based on the waveforms, makes a biometric identification of the user. In some implementations, the sensor system also performs feature analysis to extract other information from the waveforms, such as posture, gender, weight and age of the user.

In illustrative implementations, the sensor system is dynamically adaptable. Among other things, the sensor system: (a) dynamically adjusts the weight given to data measured by different types of sensors (accelerometer, gyroscope, optical sensor) or (b) dynamically adjusts the weight given to data gathered at different body locations (e.g., wrist-worn, head-mounted, or handheld).

In illustrative implementations, this dynamic adjustment of weighting depends on one or more of the following trigger factors: quality of data, posture of user (e.g., standing, sitting, or lying down), identity of user, gender of user, weight of user, age of user, activity of user (e.g., reading, listening to music, talking on a phone, or browsing the Internet), availability of data, and purpose for which data is being used (e.g., whether the data is being used to calculate posture, biometric identification, heart rate, or respiratory rate).

This dynamic adjustment is advantageous because which type of sensor (e.g., accelerometer, gyroscope, or PPG sensor) or combination of types of sensors yields the most accurate estimate of a physiological parameter (or yields the most accurate biometric identification) varies under different conditions. These different conditions include: (a) the position of the sensor relative to the user's body (e.g., head-mounted, wrist worn, or carried in a pocket of the user); and (b) the trigger factors mentioned in the preceding paragraph. By dynamically adjusting the weight of data from different sensors, the sensor system is able to achieve more accurate estimates.

For example, in some cases, data from a wrist-mounted gyroscope yields a more accurate estimate of breathing rate than either (i) data from a wrist-mounted accelerometer, or (ii) data from the two sensors combined. Also, for example, in some cases, the accuracy of an estimate of heart rate or breathing rate based on data from a given sensor type (e.g., accelerometer, gyroscope, or PPG sensor) varies dramatically depending on the user's posture (e.g., whether the user is standing, sitting or supine). Similarly, in some cases, the accuracy of a biometric identification based on sensor data from an accelerometer, gyroscope or PPG sensor varies sharply, depending on the user's posture.

In some cases, the sensors include a video camera that measures motion of the user relative to a scene. In some cases, the camera is located in a fixed position in the user's surrounding and captures images of the user, from which images motion of the user is measured. In some other cases, the camera is mounted on the user and captures images of the user's surrounding, from which motion of the user is measured (by assuming that the scene captured in the image is static). Data from the video camera is also dynamically weighted, depending on factor such as (in the case of a camera worn or mounted on a user), where the camera is mounted or worn on the user.

The description of the present invention in the Summary and Abstract sections hereof is just a summary. It is intended only to give a general introduction to some illustrative implementations of this invention. It does not describe all of the details and variations of this invention. Likewise, the descriptions of this invention in the Field of Technology section and Field Of Endeavor section are not limiting; instead they each identify, in a general, non-exclusive manner, a technology to which exemplary implementations of this invention generally relate. Likewise, the Title of this document does not limit the invention in any way; instead the Title is merely a general, non-exclusive way of referring to this invention. This invention may be implemented in many other ways.

### BRIEF DESCRIPTION OF THE DRAWINGS

FIGS. 1A, 1B, and 1C each show sensor modules worn or carried at the following positions on a person's body: head, wrist, and pocket. In FIG. 1A, the person is standing. In FIG. 1B, the person is sitting. In FIG. 1C, the person is lying down.

FIG. 2 shows hardware components of a sensor system for detecting physiological parameters.

FIG. 3 shows a head-mounted sensor module, which is attached to a head-band.

FIG. 4 shows a head-mounted sensor module, which is housed in eyeglass frames.

In FIGS. 5, 6, 7, 8, 9, and 10, a sensor system includes three sensor modules: a head-mounted sensor module, a wrist-worn sensor module and a pocket sensor module. Each of these modules includes multiple sensors. Specifically: (a) each sensor module includes an accelerometer and a gyroscope, and (b) the wrist-worn sensor module also includes a photoplethysmographic (PPG) sensor.

FIGS. 5 and 6 illustrate different methods of aggregating data.

In FIG. 5, separate heart rate estimates are calculated for each sensor module. Then these estimates are aggregated.

In FIG. 6, separate heart rate estimates are calculated for each sensor. The heart rate estimates for the sensors in each sensor module are then aggregated. Then these aggregates for the sensor modules are aggregated.

FIGS. 7, 8, 9 and 10 each show an example of weighting of sensor readings. In each of FIGS. 7, 8, 9 and 10, heart rate is estimated. To calculate this estimate, data from different sensors is weighted differently. The weighting is determined by trigger factors, such as the quality of data and the posture, identity, gender, age, weight and type of activity of a subject.

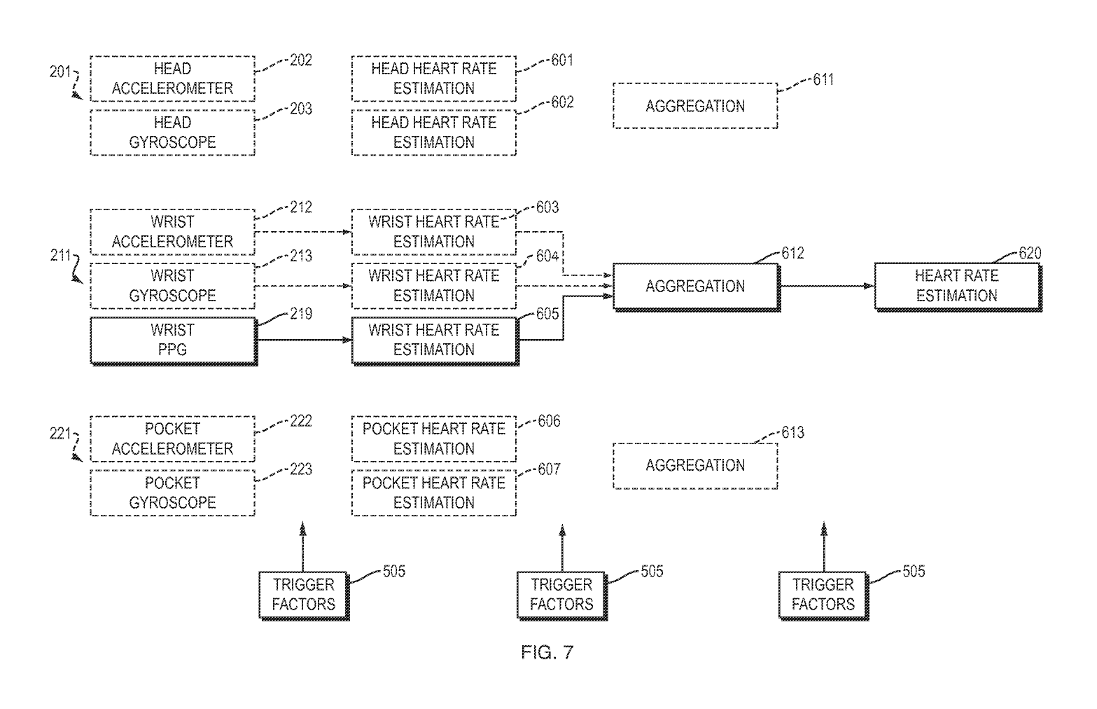

In FIG. 7, heart rate is estimated based on measurements taken only by the PPG sensor in the wrist-worn module. Readings from other sensors in the wrist-worn module and in other modules are disregarded.

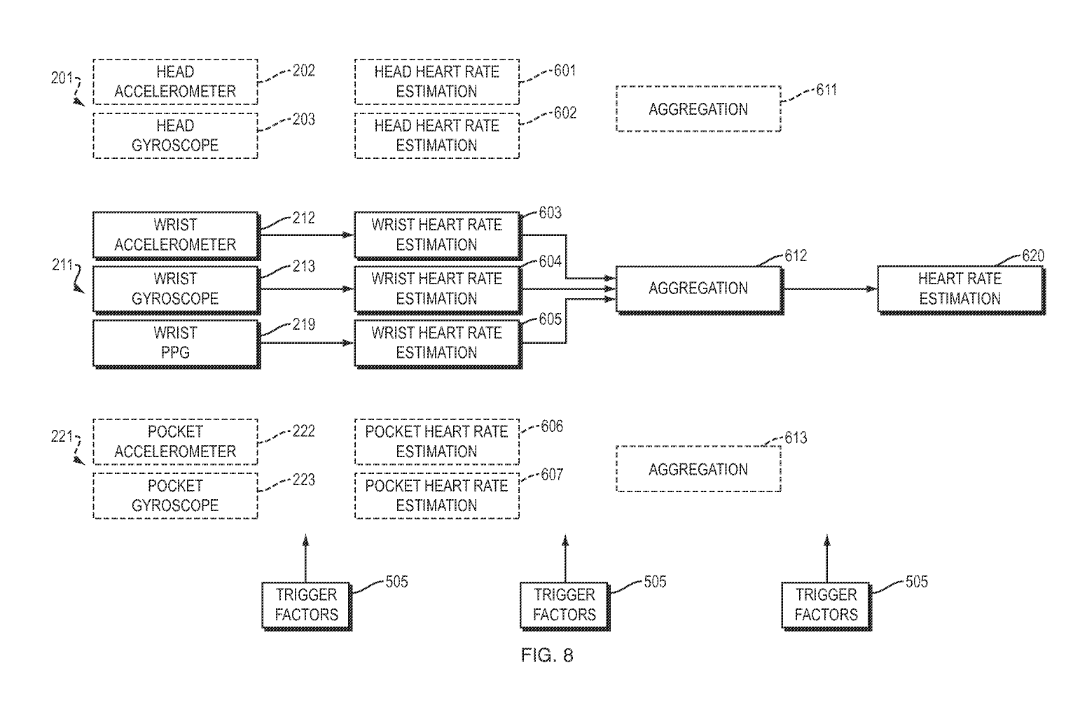

In FIG. 8, separate heart rate estimates are calculated for each sensor in the wrist-worn module. Then these heart rate estimates are aggregated. Readings from other sensor modules are disregarded.

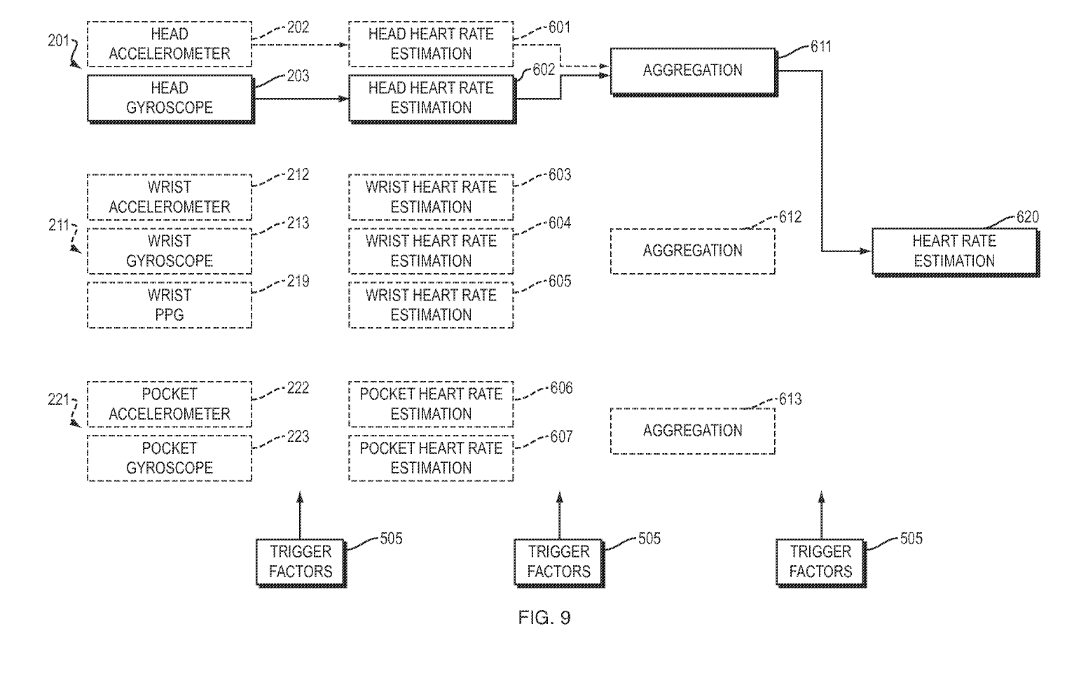

In FIG. 9, heart rate is estimated based on measurements taken only by the gyroscope in the head-mounted module. Readings from other sensors in the head-mounted module and in other modules are disregarded.

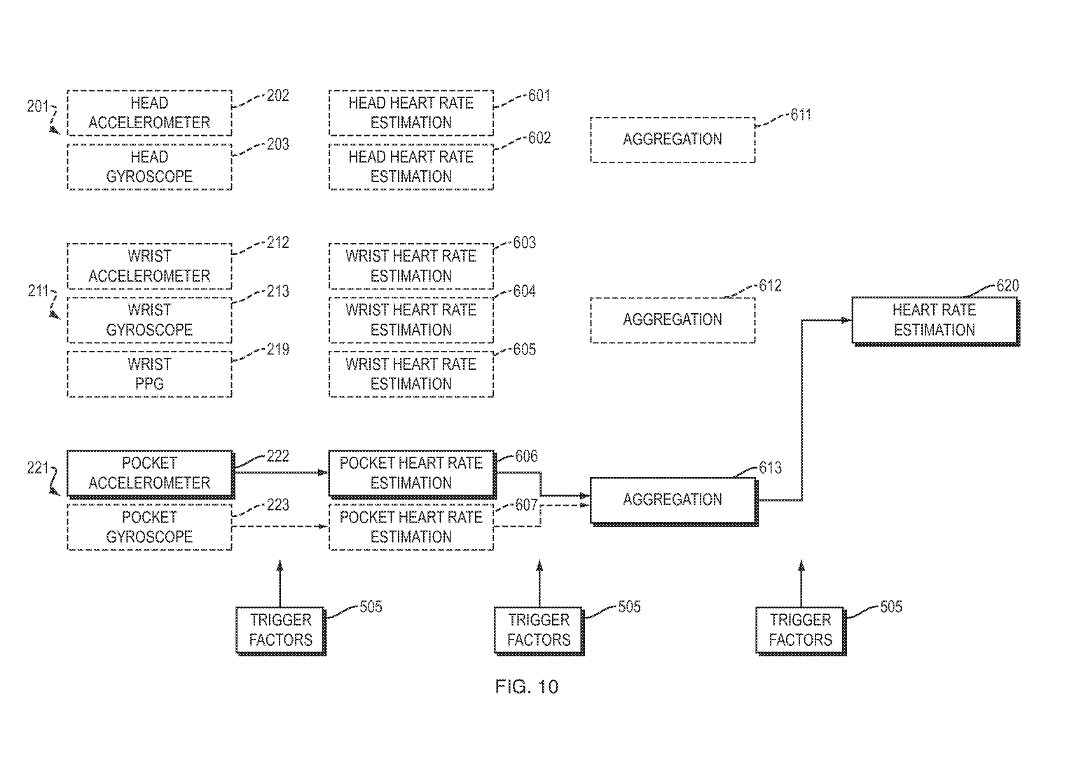

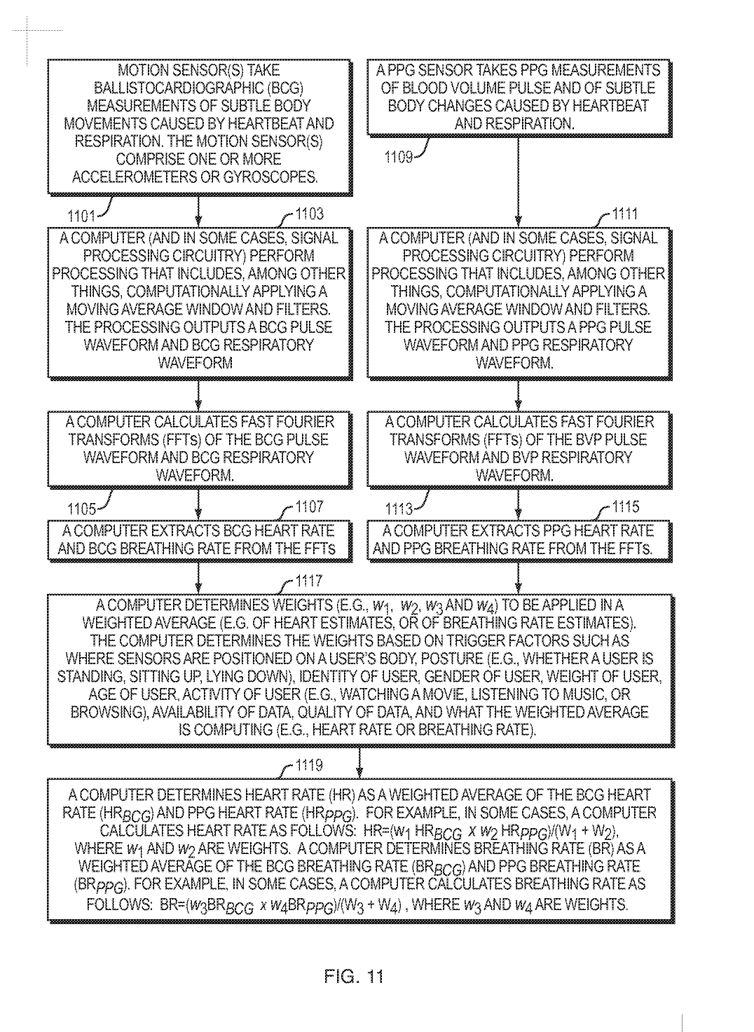

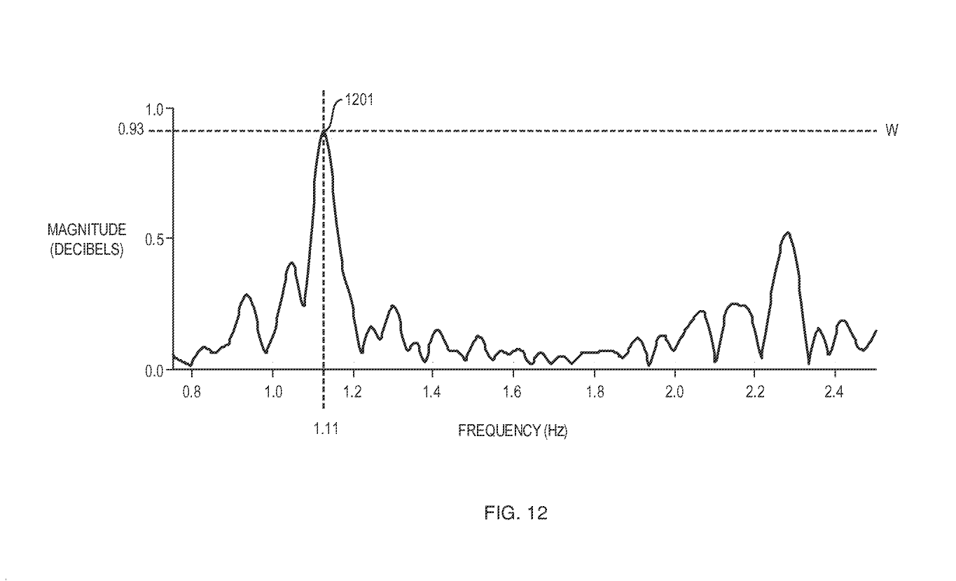

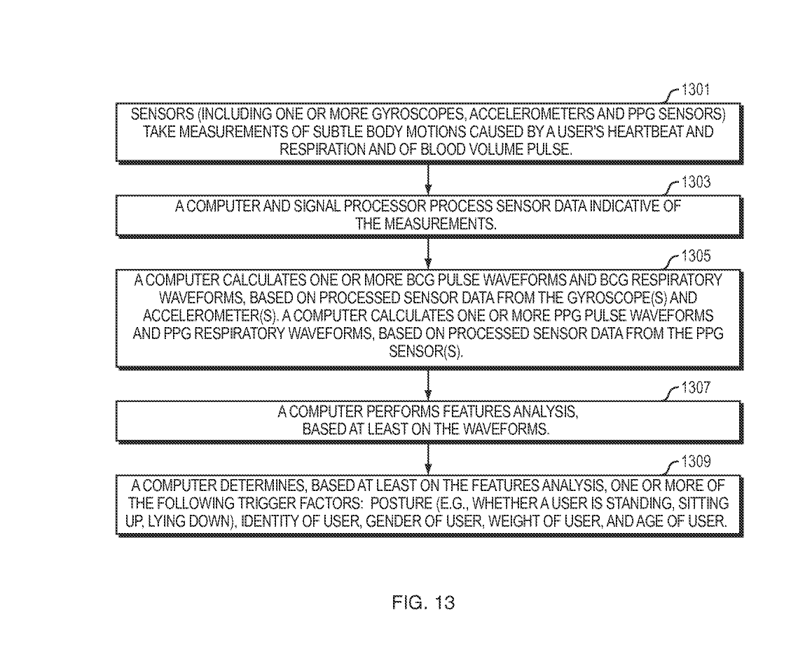

In FIG. 10, heart rate is estimated based on measurements taken only by the accelerometer in the pocket module. Readings from other sensors in the pocket module and in other modules are disregarded.

FIG. 11 illustrates a method of determining (1) heart rate as a weighted average of a ballistocardiography (BCG) heart rate estimate and a PPG heart rate estimate, and (2) breathing rate as a weighted average of (i) a breathing rate estimate from accelerometer and gyroscope data and (ii) a PPG breathing rate estimate.

FIG. 12 shows an example of weighting based on magnitude of the highest magnitude frequency component.

FIG. 13 shows steps in a method of determining trigger factors by feature analysis of waveforms.

The above Figures show some illustrative implementations of this invention, or provide information that relates to those implementations. However, this invention may be implemented in many other ways.

### DETAILED DESCRIPTION

### Different Types of Sensors, and Different Sensor Positions

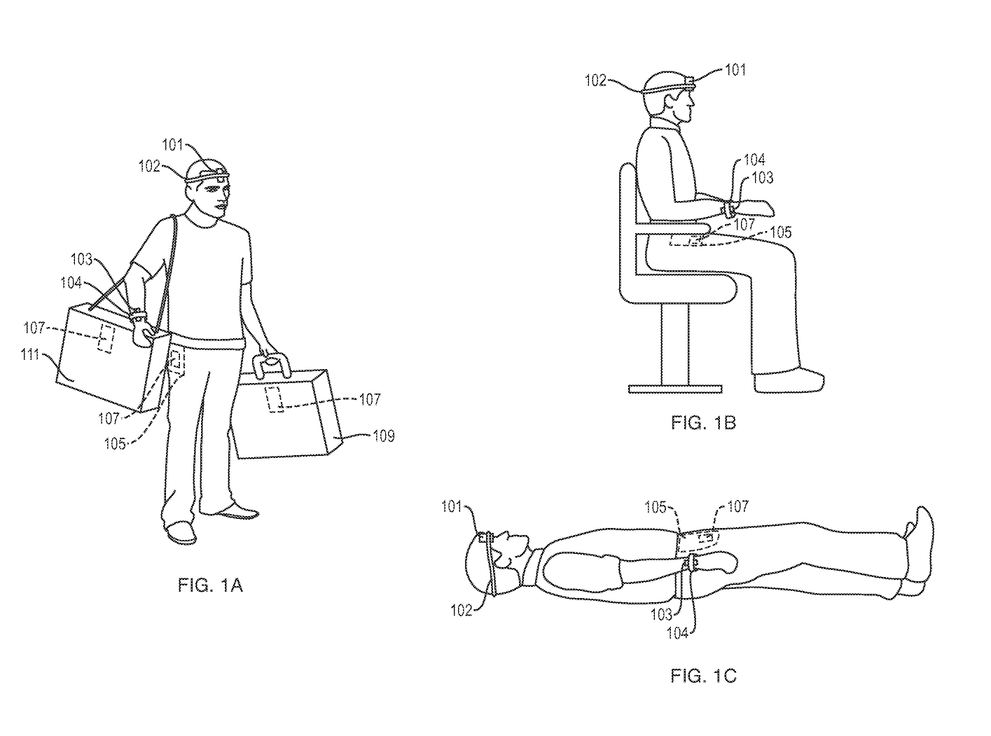

FIGS. 1A, 1B, and 1C each show sensor modules worn or carried by a person at head, wrist and pocket positions, in an illustrative implementation of this invention. The person is shown in three different body postures: standing, sitting, and supine. In FIG. 1A, the person is standing. In FIG. 1B, the person is sitting. In FIG. 1C, the person is lying down.

In FIGS. 1A, 1B, and 1C, a sensor system for detecting physiological parameters comprises three sensor modules: (a) a head-mounted sensor module 101 worn on a head-band 102; (b) a wrist-mounted sensor module 103 worn on a wrist band 104; and (c) a pocket sensor module 107 worn in a pocket 105. In some cases, pocket sensor module 107 is included in a smartphone or other mobile computing device. In some use scenarios, sensor module 107 is not carried in a pocket. For example, in some cases, sensor module 107 is a smartphone and is held by a user while reading content on a screen of the smartphone, or is carried in a handheld briefcase 109, or is carried in a briefcase (or other bag or purse) 111 supported by a strap worn over the subject's shoulder.

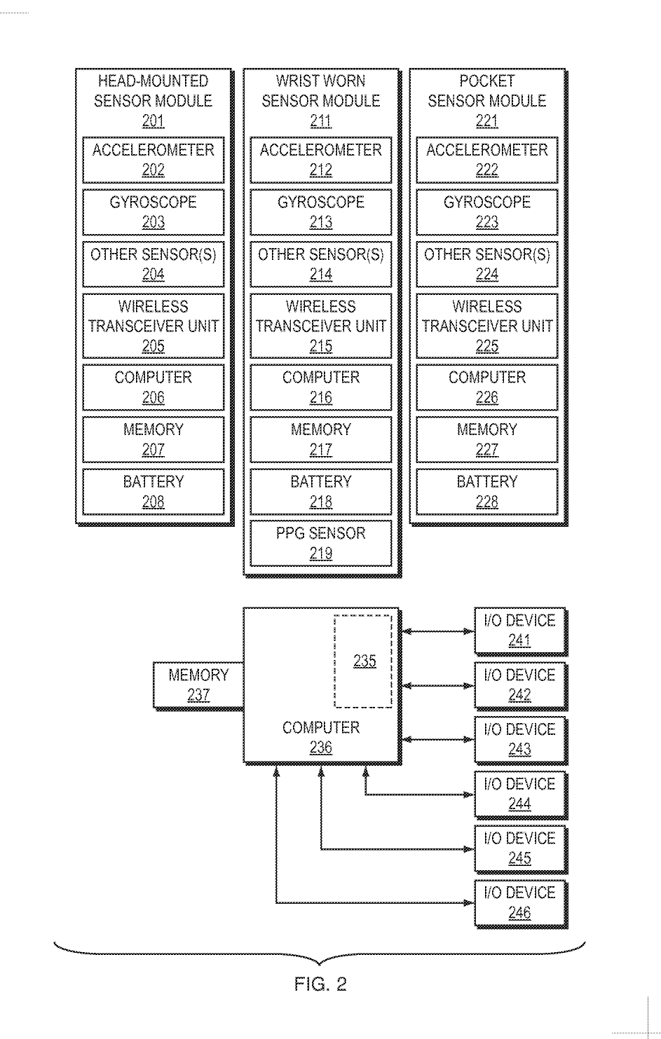

FIG. 2 shows hardware components of a sensor system for detecting physiological parameters, in an illustrative implementation of this invention. The sensor system includes three sensor modules: a head-mounted sensor module 201, a wrist-worn sensor module 211, and a sensor module 221 carried in a pocket. Each of the three sensor modules includes an accelerometer (e.g., a 3-axis digital accelerometer) 202, 212, 222, a gyroscope (e.g., a 3-axis digital gyroscope) 203, 213, 223, a wireless transceiver unit 205, 215, 225, a computer (e.g., a microcontroller) 206, 216, 226, an electronic memory device 207, 217, 227, and a battery 208, 218, 238. In addition, the wrist-worn sensor module 211 includes a PPG sensor 219. In some cases, at least one of the sensor modules include one or more other sensors 204, 214, 224, such as one or more video cameras, electrodermal activity (EDA) sensors, or thermometers. Each sensor module includes a housing that provides structural support of components (e.g., sensors) that are onboard the module. Each battery 208, 218, 228 provides power for the sensors, computer, memory device, and wireless transceiver unit in a sensor module.

In the example shown in FIG. 2, a computer 236 calculates a physiological parameter such as heart rate (HR), heart rate variability (HRV) or breathing rate (BR), based at least in part on sensor readings from the sensors or on data derived from the sensor readings. In some cases, when determining a physiological parameter (e.g., HR, HRV, or BR), the computer 236 weights input from different sensors by different amounts. The computer 236 determines the weighting based on one or more trigger factors such as sensor position relative to a user's body, posture (e.g., whether a user is standing, sitting up, lying down), identity of user, gender of user, weight of user, age of user, activity of user (e.g., watching a movie, listening to music, or browsing), availability of data, quality of data, and what the weighted average is computing (e.g., heart rate or breathing rate). The computer 236 determines the trigger factors by either: (a) accessing a database that stores user-inputted information regarding the trigger factors, (b) downloading information regarding the trigger factors from the Internet, (c) by accepting user input regarding the trigger factors, or (d) by analyzing data from one or more of the sensors. For example, a human user may input data that indicates the user's identity, age, gender or weight. Or, for example, computer 236 may process data indicative of sensor readings from one or more of the sensors, in order to calculate any one or more of the trigger factors.

In the example shown in FIG. 2, I/O devices 241, 242, 243, 244, 245, 246 accept input from a human user and output information in human-understandable format. For example, the I/O devices may comprise one or more of the following: a touch screen, other electronic display screen, keyboard, mouse, microphone, speaker, or digital stylus. Computer 236 stores data in, and accesses data from, an electronic memory device 237.

Each wireless transceiver unit (e.g., 205, 215, 225, 235) includes (a) one or more antennas, (b) one or more wireless transceivers, transmitters or receivers, and (c) signal processing circuitry. Each wireless transceiver unit receives and transmits data in accordance with one or more wireless standards.

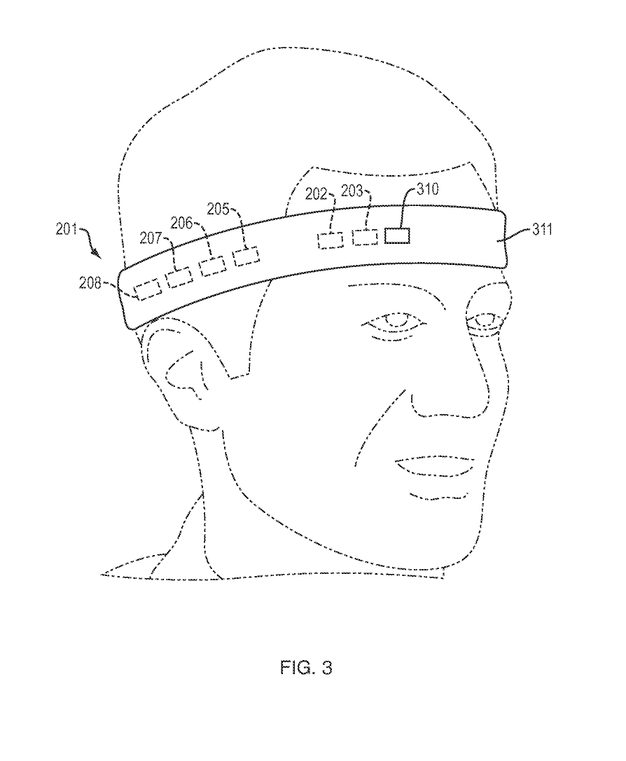

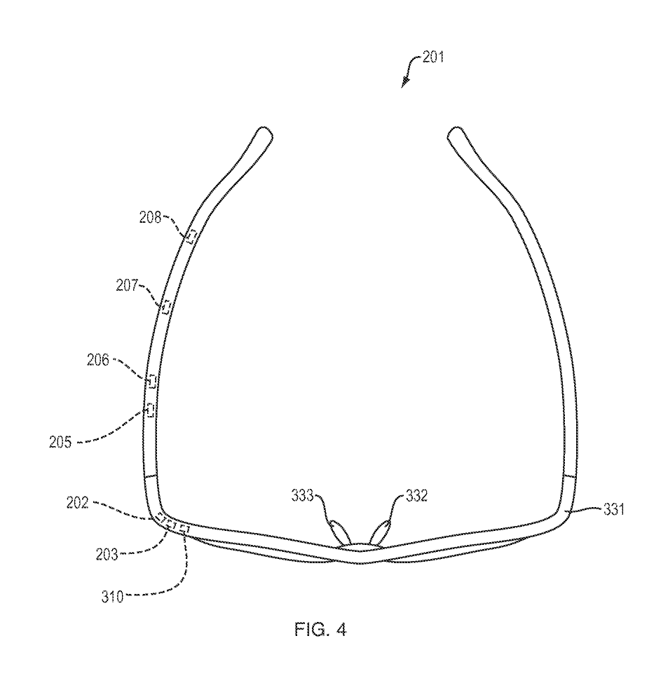

FIG. 3 is a perspective view of a head-mounted sensor module 201 and support structure, in an illustrative implementation of this invention. FIG. 4 is a top view of another example of a head-mounted sensor module and support structure. In FIGS. 3 and 4, the sensor module includes a gyroscope, accelerometer and camera.

In the examples shown in FIGS. 3 and 4, an accelerometer 202, gyroscope 203, camera 310, wireless transceiver unit 205, computer 206, electronic memory device 207 and battery 208 are housed in, or permanently or releasably attached to, a support structure.

A wide variety of cameras may be used. For example, in some cases, the camera 310 is a video camera. In some cases, the camera is a depth-sensing camera, including a depth-sensing video camera. In other cases, the camera is an infra-red camera, including an infra-red video camera.

A wide variety of support structures may be used.

In the example shown in FIG. 3, the support structure comprises elastic headware 311. In some cases, the elastic headwear 311 comprises a material that stretches (elastically deforms). In some cases, this headwear 311, when elastically deformed, has a length, around a circumference or perimeter of the headware (or around the edge of a hole formed by the headware) that: (a) is in a range between 50 cm and 65 cm, and thus is configured to fit snugly around an adult human head; or (b) is in a range between 40 cm and 55 cm, and thus is configured to fit snugly around a child's head; or (c) is in a range between 32 cm and 52 cm, and thus is configured to fit snugly around a head of a human who is between zero and 36 months old. For example, in some cases, the elastic headwear 311 comprises (i) a headband, or (ii) elastic apparel that has a convex shape that fits on or over (or partially surrounds or conforms to the shape of) a human head.

More generally, the support structure comprises any headwear, including: (a) any hat, cap, helmet, eyeglasses frame, sunglasses frame, visor, headband, crown, diadem, or head-mounted display, or (b) any structure (including any strap, band, frame, ring, post, scarf, or other item of apparel) that is worn at least partially on or supported at least partially by the skin, hair, nose or ears of a human head or that at least partially surrounds or indirectly rests upon a human neurocranium. However, the term “headwear” does not include any part of a human being.

In the example shown in FIG. 4, at least a portion of support structure 331 is rigid. In some cases, support structure 331 includes joints or hinges, such that rigid portions of structure 331 may rotate about the joint or hinge. Support structure 331 is configured to rest upon protuberances of a human head. Specifically, support structure 331 is configured to rest upon, and be supported by, the ears and nose of a human user. For example, support structure 331 includes two nosepads 332, 333. Support structure 331 is similar in shape to, or is part of, of an eyeglasses frame.

In the example shown in FIGS. 3 and 4, a computer 206 processes sensor data gathered by the accelerometer 202, gyroscope 203, and camera 310. In some cases, the computer 206 comprises a microprocessor. The computer 206 stores data in, and reads data from, the memory device 207. The computer 206 communicates with remote devices via a wireless transceiver unit 205.

In some cases, the wrist-mounted sensor module is housed in a smartwatch, which includes a watch band worn around a user's wrist. Alternatively, the wrist-mounted sensor module is housed or attached to an elastic wristband or to a wristband that has an adjustable inner diameter. For example, in some cases, the inner diameter of the wristband may be adjusted by inserting a metal prong attached to one end of the wristband into one of a series of holes at the other end of the wrist band.

In some cases, the wrist-mounted sensor module includes a PPG sensor. The PPG sensor includes an LED (light emitting diode) that illuminates a user's skin, and a photodiode that measures the amount of light that is transmitted through or reflected from the user's skin.

A computer (e.g., computer 236) uses sensor data, or data derived therefrom, to calculate HR, HRV and BR. Alternatively, a computer (e.g., 206, 216, 226) onboard a sensor module calculates HR, HRV and BR.

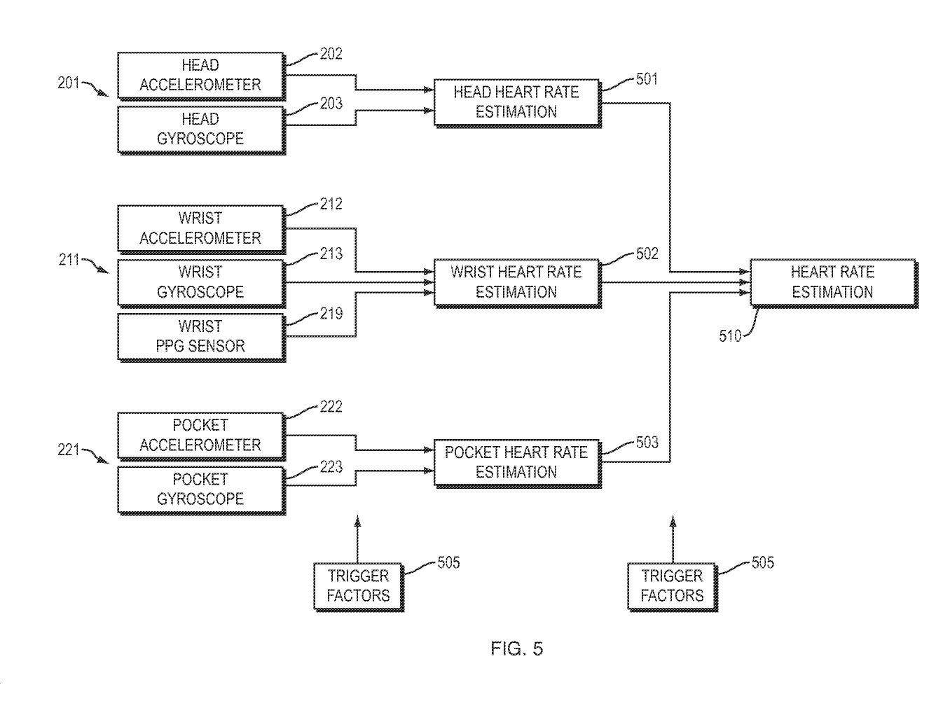

In FIGS. 5, 6, 7, 8, 9, and 10, a sensor system includes three sensor modules: a head-mounted sensor module 201, a wrist-worn sensor module 211 and a pocket sensor module 221. Each of these modules includes multiple sensors. For example: (a) each sensor module includes an accelerometer and a gyroscope, and (b) the wrist-worn sensor module 211 also includes a photoplethysmographic (PPG) sensor.

In illustrative implementations, a multi-sensor wireless communications network collects data from sensors worn or carried at different locations relative to the user's body to determine the best estimate of the heart-rate or respiration. In addition to, or instead of, one or more of the locations discussed above (head, wrist, pocket), the sensor modules may be worn on the lower calf, worn above the ankle, clipped onto a belt, or worn or carried anywhere else on the body. Each of the sensor modules includes one or more sensors (such as a 3-axis digital accelerometer, a 3-axis digital gyroscope and a PPG sensor) for detecting blood volume pulse or subtle bodily motion caused by heartbeat or respiration.

Calculating Pulse Waveform from Motion Sensor Readings

In some cases, measurements taken by a 3-axis motion sensor (e.g., a 3-axis accelerometer or 3-axis gyroscope) are used to compute a cardiac pulse waveform as follows: First, represent each sample taken by the 3-axis motion sensor as a 3D vector, where the first, second and third elements of the vector are x-axis, y-axis and z-axis measurements, respectively, for that sample. A time sequence of samples by the 3-axis motion sensor is represented by a time series of 3D vectors or, equivalently, by a matrix in which the first, second and third rows represent x-axis, y-axis and z-axis measurements, respectively, and each column represents a sample at a particular time. In this matrix, each row represents a time sequence of readings taken by a particular axis (x, y or z) of the motion sensor. Second, for each row, subtract a moving average window from each entry in the row to detrend readings and to filter artifacts caused by sudden, large movements. The window size depends on the sampling rate of the motion sensor. For example, in some cases: (a) the sampling rate of the motion sensor is 256 Hz; (b) the window size is 15 samples; (c) for each given entry in a row, the values of the given entry and the surrounding 14 entries (7 before and 7 after) in the row are averaged, then the average is subtracted from the given entry. Near the beginning and end of a row, if the number of subsequent entries in the row is less than the window size divided by two, then the window size is reduced in one of the sides. Third, z-score each row of the matrix to have zero mean and unit variance, so that the axes have the same relevance and the analysis is more robust to changing orientations of the motion sensor. Fourth, apply a band-pass Butterworth filter (e.g. with cut-off frequencies of 7 Hz and 13 Hz) to each axis (row of the matrix). Fifth, in order to aggregate the different components (i.e., axes of the motion sensor), compute the square root of the summation of the squared components (i.e., L2 norm) at each sample. The output of this aggregation is a 1D vector, where each entry in the 1D vector represents an aggregated value for all three axes for a given sample. Sixth, apply another Butterworth bandpass filter (e.g., with cut-off frequencies of 0.66 Hz and 2.50 Hz), in order to compute the pulse waveform.

In illustrative implementations, the Butterworth filters (e.g., used in computing pulse waveforms or respiratory waveforms from accelerometer, gyroscope or PPG sensor readings) are applied computationally. The order of the Butterworth filters may vary, for example from first order to fourth order. In many cases, a higher order (e.g., 4) Butterworth is desirable for better filtering. However, in some cases, a lower order Butterworth filter (e.g., first or second order) is desirable because of a low signal amplitude or in order to simplify computation.

Calculating Respiratory Waveform from Motion Sensor Readings

In some cases, measurements taken by a 3-axis motion sensor (e.g., a 3-axis accelerometer or 3-axis gyroscope) are used to compute a respiratory waveform as follows: First, represent each sample taken by the 3-axis motion sensor as a 3D vector, where the first, second and third elements of the vector are x-axis, y-axis and z-axis measurements, respectively, for that sample. A time sequence of samples by the 3-axis motion sensor is represented by a time series of 3D vectors or, equivalently, by a matrix in which the first, second and third rows represent x-axis, y-axis and z-axis measurements, respectively, and each column represents a sample at a particular time. In this matrix, each row represents a time sequence of readings taken by a particular axis (x, y or z) of the motion sensor. Second, for each row, subtract a moving average window (e.g., with a window size of 8.5 seconds) from each entry in the row. Near the beginning and end of a row, if the number of subsequent entries in the row is less than the window size divided by two, then the window size is reduced. Third, z-score each axis (row of the matrix) to have zero mean and unit variance. Fourth, apply Independent Components Analysis (ICA) to each axis (row of the matrix) to determine independent components of the axis and remove sources of artifact. Fifth, to isolate movements caused by respiration, apply a band-pass Butterworth filter (e.g. with cut-off frequencies of 0.13 Hz and 0.66 Hz) to each independent component. Sixth, calculate Fast Fourier Transforms (FFTs) for each independent component of each axis. Seventh, determine which FFT (out of the FFTs of independent components of all of the axes) is the most periodic—i.e., which FFT has the maximum magnitude. Seventh, select the independent component that has the most periodic FFT within a specific frequency range (e.g. 0.13 Hz and 0.66 Hz), and output it as the respiratory waveform (or output its FFT as the FFT of the respiratory waveform). Alternatively: (a) in the fourth step, use Principal Components Analysis (PCA) instead of ICA; and (b) substitute principal components for independent components in the remainder of the above steps.

Calculating Pulse and Respiratory Waveforms from Other Sensor Readings

In some cases: (a) a sensor module includes a PPG sensor; and (b) readings from the PPG sensor are processed and filtered in order to extract pulse and respiratory waveforms. Likewise, in some cases: (b) a sensor module includes a video camera or other sensor; and (b) readings from the camera or other sensor are processed and filtered in order to extract pulse and respiratory waveforms.

Extracting Physiological Parameters from Pulse and Respiratory Waveforms

In illustrative implementations, a computer extracts heart rate from the pulse waveform. To do so, a computer computes FFTs of the pulse waveform and analyzes the frequency response in a range from 0.66 Hz-2.5 Hz (corresponding to 45 to 150 beats per minute). The computer identifies the frequency component in that range that has the highest amplitude, and then multiplies that frequency (which is expressed in Hertz) by 60 seconds/minute, in order to output a heart rate expressed as beats per minute.

In illustrative implementations, a computer extracts breathing rate form the respiratory waveform. To do so, a computer computes a FFT of the respiratory waveform (or, if the FFT of the respiratory waveform was computed in an earlier step, uses that FFT). The computer analyzes the frequency response in a range from 0.13 Hz to 0.66 Hz (corresponding to 8 to 45 breaths per minute). The computer identifies the frequency component in that range that has the highest amplitude, and then multiplies that frequency (which is expressed in Hertz) by 60 seconds/minute, in order to output a breathing rate expressed as breaths per minute.

### Sampling Rate

The sampling rate of the motion sensors (and any other sensors used to calculate a physiological parameter) may vary, depending on the particular implementation or use scenario. In some cases, sensor data is stored for long periods onboard a sensor module before being transmitted for further analysis, and thus a low sampling rate (e.g., between 20 Hz and 50 Hz) is desirable. In some other cases, a higher sampling rate (e.g., between 100 Hz and 256 Hz, or higher than 256 Hz) is desirable, such as for calculating heart rate variability, for which a high temporal resolution is advantageous.

### Dynamic Weighting of Sensor Measurements or of Data Derived Therefrom

In illustrative implementations, a sensor system includes one or more accelerometers, gyroscopes and optical sensors. The sensor system (a) takes sensor measurements of a human user; (b) based on the sensor measurements, calculates a cardiac waveform and a respiratory waveform; (c) based on the waveforms, calculates heart rate, breathing rate and other physiological parameters, such as heart rate variability; and (d) based on the waveforms, makes biometric identifications of humans. In some implementations, the sensor system also performs feature analysis to extract other information from the waveforms, such as posture, gender, weight and age of the user.

In illustrative implementations, the sensor system is dynamically adaptable. Among other things, the sensor system: (a) dynamically adjusts the weight given to data measured by different sensors (accelerometer, gyroscope, optical sensor) or (b) dynamically adjusts the weight given to data gathered at different body locations (e.g., wristworn, headmounted, or handheld).

In illustrative implementations, this dynamic adjustment of weighting depends on one or more of the following trigger factors: sensor position (e.g., head, wrist, pocket), quality of data, posture of user, identity of user, gender of user, weight of user, age of user, activity of user, availability of data, and purpose for which data is being used (e.g., whether the data is being used to calculate posture, biometric identification, heart rate, or respiratory rate).

In illustrative implementations, the weights given to data from each of the sensors is adjusted based on one or more trigger factors. Here are some non-limiting examples:

When both optical (e.g., PPG) sensors and motion-based sensors (e.g., accelerometer, gyroscope) are positioned on the wrist, the sensor system selects a weighted set of one or more sensors that works best for each posture. For instance, in some cases, PPG sensors outperform motion-based sensors when estimating heart rate during sitting down and lying down, and a combination of both types of sensors outperforms each of them alone when standing.

When considering sensors from different body locations, the weights are automatically selected to choose the most advantageous combination. For instance, in some cases, among several motion-based sensors on the head, a gyroscope receives a higher weight than an accelerometer. However, in some cases, when gathering the same sensor information from a smartphone carried in a pocket, the accelerometer receives a higher weight than the gyroscope.

Different sensors receive different weights based on the quality and availability of the data. For instance, if the person carries devices on the head and the wrist, the algorithm will dynamically select the one that contains less noise and best signal to noise ratio. For instance, while typing at the computer, head sensors are expected to have less noise than wrist sensors.

In some implementations, posture is the only trigger factor that controls the dynamic adjustment of weighting. In some other implementations, identity or activity of a user is the only trigger factor that controls weighting. In other more complicated versions, there are multiple trigger factors that interact in more complex and perhaps iterative ways (see list of trigger factors above).

The weighting of sensors (by different sensor type or by different sensor position relative to the body) may be done in different ways, in illustrative implementations of this invention. In some cases, the signal stream for each sensor, respectively, is assigned a weight, so that data from different sensors may have different weights. In some other cases, an intermediate output of processing (e.g., pulse and respiratory waves, heart and breathing rates) performed by each of the sensors are weighted and combined. Both approaches may also be combined. The weighting may be done with different operators such as a weighted average (e.g., continuous weights) or filtering approach (e.g., binary weights).

In some cases, weights are determined in advance, in either a person-independent or a person-dependent way, using machine learning to infer an optimal method of combining the observations from the sensors being worn. For example, a Bayesian fusion method or a decision-tree may be trained on prior data and the weights applied to current data streams. Many types of machine learning may be used; this invention is not limited to a particular type. In many cases, the weighting is dependent not only on the type of sensors and their placement and on other fixed properties (e.g., gender or posture), but also on dynamic qualities of the current data and noise stream (e.g. data indicating that a car is accelerating).

In some cases, physiological parameters (e.g., HR or BR) are estimated separately by each sensor and then a weighted average of the estimates is taken, where the weight assigned to each estimate is equal to the magnitude of the highest magnitude frequency component of the waveform used to calculate the estimate For instance, in some cases, in order to aggregate readings by motion-based and optical sensors on the wrist, a weighted combination of the HR estimates obtained by the two types of sensors is calculated, as follows: (a) the heart rate (HR) measured by the motion-based sensor is estimated as the frequency of the highest magnitude component of the FFT of the pulse waveform measured by the motion-based sensor; (b) the HR measured by the optical sensor is estimated as the frequency of the highest magnitude component of the FFT of the pulse waveform measured by the optical sensor; (c) the motion-based HR estimate is weighted by a first weight equal to the magnitude of the highest magnitude component of the FFT of the cardiac waveform measured by the motion-based sensor; (d) the optical HR estimate is weighted by a second weight equal to the magnitude of the highest magnitude component of the FFT of the cardiac waveform measured by the optical sensor; and (e) the weighted average estimate of heart rate is equal to the sum of the weighted HR estimates divided by the sum of the weights. For instance, if the highest magnitude component of the FFT of a waveform measured by a motion-based sensor occurs at 1 Hz (60 beats per minute) with a magnitude of 0.5 dB and if the highest magnitude component of the FFT of a waveform measured by an optical sensor occurs at 1.5 Hz (90 beats per minute) with a magnitude of 1 dB, then a weighted average estimate of heart rate, weighted according to these magnitudes, is 1.33 Hz, corresponding to 80 beats per minute—that is ((1 Hz×w₁)+(1.5 Hz×w₂))/(w₁+w₂)=1.33 Hz, where w₁=0.5 and w₂=1.0.

As used herein: (a) a “head heart rate estimation” means an estimation of heart rate from measurements taken only by one or more sensors in a head-mounted sensor module 201; (b) a “wrist heart rate estimation” means an estimation of heart rate from measurements taken only by one or more sensors in a wrist-worn sensor module 211; (c) a “pocket heart rate estimation” means an estimation of heart rate from measurements taken only by sensors in a sensor module 221 worn in a pocket; (d) “head accelerometer” means a 3-axis accelerometer (e.g., 202) in a head-mounted sensor module 201; (e) “head gyroscope” means a 3-axis gyroscope (e.g., 203) in a head-mounted sensor module 201; (f) “wrist accelerometer” means a 3-axis accelerometer (e.g., 212) in a wrist-worn sensor module 211; (g) “wrist gyroscope” means a 3-axis gyroscope (e.g., 213) in a wrist-worn sensor module 211; (g) “wrist PPG” means a PPG sensor (e.g., 219) in a wrist-worn sensor module 211; (h) “pocket accelerometer” means a 3-axis accelerometer (e.g., 222) in a sensor module 221 worn in a pocket; and (h) “pocket gyroscope” means a 3-axis gyroscope (e.g., 223) in a sensor module 221 worn in a pocket.

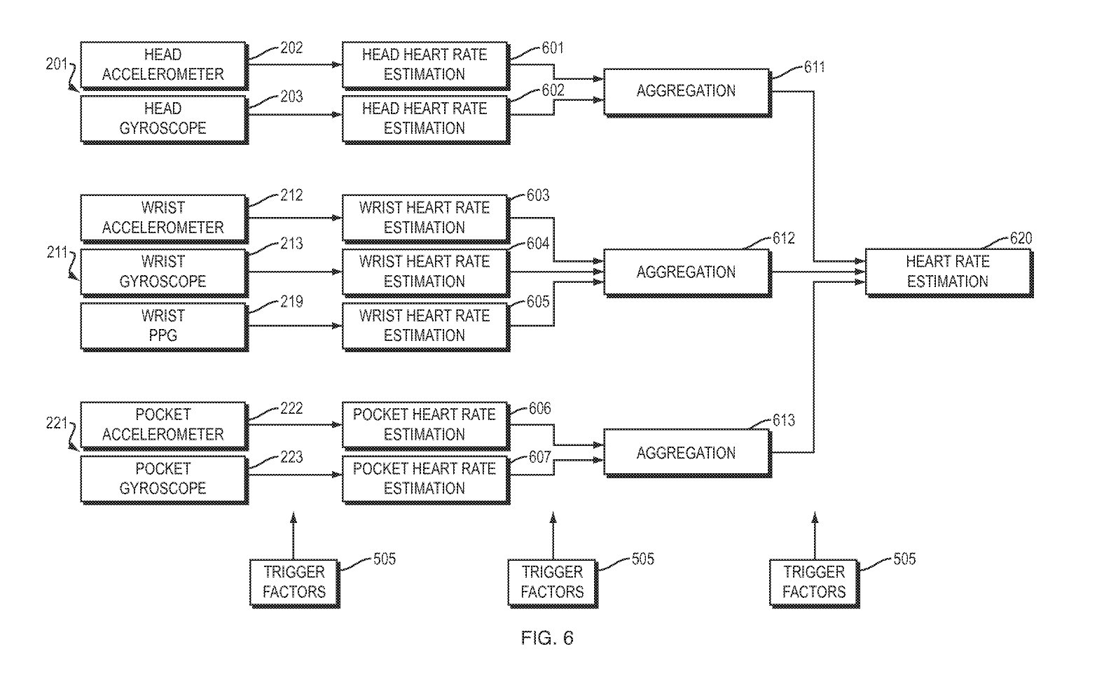

FIGS. 5 and 6 illustrate different methods of aggregating data, in an illustrative implementation of this invention.

In FIG. 5, separate heart rate estimates are calculated for each sensor module. Then these estimates are aggregated.

Specifically, in the example shown in FIG. 5: (a) Head heart rate estimation 501 is calculated in a computation that includes sensor readings from the head accelerometer 202 and the head gyroscope 203; and (b) pocket heart rate estimation 503 is calculated in a computation that includes components of sensor readings from the pocket accelerometer 222 and pocket gyroscope 223. For each of these two cases, the sensor streams are aggregated into a single matrix in which the first, second and third rows represent x-axis, y-axis and z-axis measurements of accelerometers, respectively, and the fourth, fifth and sixth rows present x-axis, y-axis and z-axis measurements of gyroscopes, respectively. Thus, each row of the matrix represents a time sequence of readings taken by a particular axis (x, y or z) of the motion sensors.

In FIG. 5, wrist heart rate estimation 502 is calculated by aggregating components of sensor readings from wrist accelerometer 212, wrist gyroscope 213 and wrist PPG sensor. In this case, motion-based components (gyroscope and accelerometer) are aggregated into a single matrix in which the first, second and third rows represent x-axis, y-axis and z-axis measurements of accelerometers, respectively, and the fourth, fifth and sixth rows present x-axis, y-axis and z-axis measurements of gyroscopes, respectively. Thus, each row of the matrix represents a time sequence of readings taken by a particular axis (x, y or z) of the motion sensors. From this matrix, pulse and respiratory waves are extracted. In order to combine these with the optical-based measurements (PPG), a weighted combination of heart and breathing rates is performed. The weights may be determined based on frequency analysis of the signals as described above.

In some cases, in order to aggregate components in FIG. 5, measurements taken the sensors in a single sensor module are synchronized, so that each sensor takes a sample at the same time. In FIG. 5, if the sampling rates of the sensors in a sensor module would otherwise be different, then samples are extrapolated to the higher sampling in order to have a uniform sampling rate for all sensors in a given sensor module.

In FIG. 5, after the head heart rate estimation 501, the wrist heart rate estimation 502, and the pocket heart rate estimation 503 are calculated, then heart rate estimation 510 is calculated, as a weighted average of estimates 501, 502, 503. A computer may dynamically vary the weighting in this weighted average over time, in response to different trigger factors.

As used herein, a “heart rate estimation” means an estimate of heart rate, and is not merely (a) a pulse waveform, or (b) components used to compute a pulse waveform.

In FIG. 6, separate heart rate estimates are calculated for each sensor. The heart rate estimates for the sensors in each sensor module are then aggregated. Then these aggregates for the sensor modules are aggregated.

Specifically, in the example shown in FIG. 6: (a) head heart rate estimation 601 is calculated from measurements taken only by head accelerometer 202; (b) head heart rate estimation 602 is calculated from measurements taken only by head gyroscope 203; (c) wrist heart rate estimation 603 is calculated from measurements taken only by wrist accelerometer 212; (d) wrist heart rate estimation 604 is calculated from measurements taken only by wrist gyroscope 213; (e) wrist heart rate estimation 605 is calculated from measurements taken only by wrist PPG 219; (f) pocket heart rate estimation 606 is calculated from measurements taken only by pocket accelerometer 222; and (g) pocket heart rate estimation 607 is calculated from measurements taken only by pocket gyroscope 223. Then: (a) a first aggregate estimate 611 is calculated by taking a weighted average of the two head heart rate estimations 601, 602; (b) a second aggregate estimate 612 is calculated by taking a weighted average of the three wrist heart rate estimations 603, 604, 605; and (c) a third aggregate estimate 613 is calculated by taking a weighted average of the two pocket heart rate estimations 606, 607. Then, a heart rate estimate 620 is calculated by taking a weighted average of the first, second and third aggregate estimates 611, 612, 613.

FIGS. 7, 8, 9 and 10 each show an example of weighting of sensor readings, in an illustrative implementation of this invention. In each of FIGS. 7, 8, 9 and 10, heart rate is estimated. To calculate this estimate, data from different sensors is weighted differently. The weighting is determined by trigger factors 505, such as position of sensor (e.g., head, wrist or pocket), posture (e.g., whether a user is standing, sitting up, lying down), identity of user, gender of user, weight of user, age of user, activity of user (e.g., watching a movie, listening to music, or browsing), availability of data, quality of data, and what the weighted average is computing (e.g., heart rate or breathing rate).

In FIG. 7, heart rate is estimated from measurements taken only by the PPG sensor 219 in the wrist-worn module. Readings from other sensors in the wrist-worn module and in other modules are disregarded. In FIG. 7, two intermediary estimates 605, 612 are calculated before calculating a heart rate estimation 620. Alternatively, in the example shown in FIG. 7, the two intermediary estimates 605, 612 are omitted, and the heart rate estimation 620 is calculated directly, from measurements taken only by PPG sensor 219.

In FIG. 8, separate heart rate estimates are calculated for each sensor in the wrist-worn module. Then these heart rate estimates are aggregated. Readings from other sensor modules are disregarded.

Specifically, in the example shown in FIG. 8: (a) wrist heart rate estimation 603 is calculated from measurements taken only by wrist accelerometer 212; (b) wrist heart rate estimation 604 is calculated from measurements taken only by wrist gyroscope 213; and (c) wrist heart rate estimation 605 is calculated from measurements taken only by wrist PPG 219. Then an aggregate estimation 612 is calculated, by taking a weighted average of wrist heart rate estimations 603, 604, 605. Then, a heart rate estimation 620 is calculated, based on aggregate estimation 612. Alternatively, in the example shown in FIG. 8, the intermediary estimate 612 is omitted, and the heart rate estimation 620 is calculated directly as a weighted average of wrist heart rate estimations 603, 604, 605.

In FIG. 9, heart rate is estimated from measurements taken only by the head gyroscope 203. Readings from other sensors in the head-mounted module and in other modules are disregarded. In FIG. 9, two intermediary estimates 602, 611 are calculated before calculating a heart rate estimation 620. Alternatively, in the example shown in FIG. 9, the two intermediary estimates 602, 611 are omitted, and the heart rate estimation 620 is calculated directly, based on measurements taken only by head gyroscope 219.

In FIG. 10, heart rate is estimated from measurements taken only by the accelerometer 222 in the pocket module. Readings from other sensors in the pocket module and in other modules are disregarded. In FIG. 10, two intermediary estimates 606, 613 are calculated before calculating a heart rate estimation 620. Alternatively, in the example shown in FIG. 10, the two intermediary estimates 606, 613 are omitted, and the heart rate estimation 620 is calculated directly, based on measurements taken only by pocket accelerometer 222.

In illustrative implementations, the sensor module includes sensors that measure different things (i.e., a gyroscope measures rotation, an accelerometer measures linear acceleration, and the camera captures visual images). Having a sensor module with different sensors that measure different things is advantageous, because in some use scenarios, large artifacts reduce the accuracy of one or two of the sensors, but not the remaining sensor(s). For example, in some cases, a computer disregards, for purposes of calculating respiration rate, cardiac pulse rate or heart rate variability, data gathered by the accelerometer during periods in which the magnitude of linear acceleration measured by the accelerometer exceeds a specified threshold. Consider the following example (the “car example”): in a rapidly accelerating car, the car's linear acceleration, in the time domain, produces a large artifact for the accelerometer, but does not affect the gyroscope. In that case, it may be desirable to disregard the accelerometer data gathered during the rapid acceleration of the car.

In the examples shown in FIGS. 7, 8, 9 and 10 and in the car example, the weighting is binary: that is, measurements taken by a particular sensor or sensor module are either given full weight (i.e., multiplied by a weight of 1) or disregarded (i.e., multiplied by a weight of zero). Alternatively, in some cases (including in alternative versions of the examples shown in FIGS. 7-10 and the car example), the weighting is not binary: that is, measurements taken by a particular sensor or sensor module are either given a zero, fractional or full weight (i.e., multiplied by a zero weight, multiplied by a fractional weight between zero and one, or multiplied by a weight of one).

In FIGS. 5, 6, 7, 8, 9 and 10 and the car example, calculations are performed by one or more computers. In some cases, a computer (e.g., 236) that is separate from the sensor modules performs these calculations. For example, in some cases: (a) a sensor module (e.g., pocket worn sensor module) is housed in a smart phone or other mobile computing device (MCD); and (b) a computer (e.g., 236) onboard the MCD performs all or part of the calculations in FIGS. 5-10. Alternatively, in some cases, computer 236 is (a) a host server connected to a network such as the Internet, or (b) is a remote personal computer. In some cases, at least a portion of the calculations in FIGS. 5-10 and the car example are performed by computers (e.g., microprocessors) 206, 216, 226 onboard the sensor modules.

In FIGS. 5, 6, 7, 8, 9, and 10, a computer determines weights to be applied to sensor readings or to information derived from the sensor readings (such as a pulse or respiratory waveform or an estimate of HR, HRV or BR or of another physiological parameter). The computer determines the weights based on trigger factors 505 such as sensor position (e.g., head, wrist or pocket), posture (e.g., whether a user is standing, sitting up, lying down), identity of user, gender of user, weight of user, age of user, activity of user (e.g., watching a movie, listening to music, or browsing), availability of data, quality of data, and what the weighted average is computing (e.g., heart rate or breathing rate). In the examples shown in FIGS. 5-10: (a) a computer dynamically adjusts a given weighted average, such that weighting in the weighted average may change over time; and (b) a computer dynamically adjusts a given weighted average, such that weighting may vary from weighted average to weighted average, and may vary over time in any given weighted average or in any given group of weighted averages. A computer determines the weighting and the dynamic adjustment of the weighting, based in part on one or more of the trigger factors.

In FIGS. 5, 6, 7, 8, 9 and 10, heart rate (i.e., cardiac pulse rate) is determined. However, other physiological parameters may be determined. For example, in some cases (including in alternative versions of FIGS. 5-10), the physiological parameter that is determined is breathing rate, heart rate variability or another cardiac parameter (such as a shape of the cardiac pulse waveform).

As used herein, the terms “ballistocardiographic” and “BCG” are adjectives that mean: of or relating to estimating pulse rate or breath rate from measurements of body movements caused by cardiac pulse or by respiration. This definition is different than the common meaning of those terms.

FIG. 11 illustrates a method of determining (1) heart rate as a weighted average of a BCG heart rate estimate and a PPG heart rate estimate, and (2) breathing rate as a weighted average of a breathing rate estimate from gyroscope and accelerometer data and a PPG breathing rate estimate, in an illustrative implementation of this invention.

In FIG. 11, the BCG heart rate estimate is determined from a BCG pulse waveform. The BCG breathing rate estimate is determined from a BCG respiratory waveform. The BCG pulse and respiratory waveforms are calculated based on sensor readings by one or more motion sensors, such as an accelerometer or gyroscope.

In FIG. 11, the PPG heart rate estimate is determined from a PPG pulse waveform. The PPG breathing rate estimate is determined from a PPG respiratory waveform. The PPG pulse and respiratory waveforms are calculated based on sensor readings by one or more PPG sensors.

In FIG. 11, motion sensor(s) take ballistocardiographic (BCG) measurements of subtle body movements caused by heartbeat and respiration. The motion sensor(s) comprise one or more accelerometers or gyroscopes (Step 1101). A computer (and in some cases, signal processing circuitry) performs processing that includes, among other things, computationally applying a moving average and filters. The processing outputs a BCG pulse waveform and BCG respiratory waveform (Step 1103). A computer calculates Fast Fourier Transforms (FFTs) of the BCG pulse waveform and BCG respiratory waveform (Step 1105). A computer extracts BCG heart rate and BCG breathing rate from the FFTs (Step 1107).

In FIG. 11, a PPG sensor takes photoplethysmographic (PPG) measurements of blood volume pulse and other subtle body changes caused by heartbeat and respiration (Step 1109). A computer (and in some cases, signal processing circuitry) performs processing that includes, among other things, computationally applying a moving average window and filters. The processing outputs a PPG pulse waveform and PPG respiratory waveform (Step 1111). A computer calculates Fast Fourier Transforms (FFTs) of the PPG pulse waveform and PPG respiratory waveform (Step 1113). A computer extracts PPG heart rate and PPG breathing rate from the FFTs (Step 1115).

In FIG. 11, a computer determines weights (e.g., w₁, w₂, w₃ and w₄) to be applied in a weighted average (e.g., of heart estimates, or of breathing rate estimates). The computer determines the weights based on trigger factors such as sensor position (e.g., head, wrist or pocket), posture (e.g., whether a user is standing, sitting up, lying down), identity of user, gender of user, weight of user, age of user, activity of user (e.g., watching a movie, listening to music, or browsing), availability of data, quality of data, and what the weighted average is computing (e.g., heart rate or breathing rate) (Step 1117).

In FIG. 11, a computer determines heart rate (HR) as a weighted average of the BCG heart rate (HR_{BCG}) and PPG heart rate (HR_{PPG}). For example, in some cases, a computer calculates heart rate as follows:

HR=(w₁HR_{BCG}×w₂HR_{PPG})/(w₁+w₂), where w₁ and w₂ are weights. A computer determines breathing rate (BR) as a weighted average of the BCG breathing rate (BR_{BCG}) and PPG breathing rate (BR_{PPG}). For example, in some cases, a computer calculates breathing rate as follows: BR=(w₃BR_{BCG}×w₄BR_{PPG})/)/(w₃+w₄), where w₃ and w₄ are weights. (Step 1119).

In some cases, a PPG sensor measures blood volume pulse and subtle changes caused by respiration. For example, in some cases, these subtle changes caused by respiration that are measured by the PPG sensor include: (a) changes in pulse wave amplitude due to the fact that blood vessels are more flexible during expiration than inspiration; (b) changes in intrathoric pressure, which in turn cause changes in pulse envelope and cardiac output, which in turn cause changes in pulse wave amplitude; and (c) decreased venous return during inspiration and increased venous return during expiration.

FIG. 12 shows an example of weighting based on magnitude of the highest magnitude frequency component. In FIG. 12, an FFT of a cardiac pulse waveform has been calculated from sensor readings by a given sensor. The highest magnitude component 1201 of the FFT occurs at 1.11 Hz with a magnitude of 0.93 dB. Thus, the estimated heart rate, calculated from the given sensor, is 1.11 Hz (that is, 67 heartbeats per minute). The weight W assigned to this heart rate estimate is 0.93, which is the magnitude of the highest magnitude component of the FFT. Thus, in a weighted average of HR estimates in which weighting is based on magnitude of the highest magnitude component of a frequency response, the HR estimate for the given sensor would be 1.11 Hz and the weight W assigned to that estimate would be 0.93.

### Trigger Factors

In illustrative implementations of this invention, trigger factors are determined in a number of different ways. The trigger factors are used to determine what weights to apply in a weighted average of estimates of a physiological parameter, such as heart rate or breathing rate.

Quality of Data: In some cases, quality of data is a trigger factor (or the sole trigger factor) that affects weighting.

If the physiological parameter being measured relates to periodic physical phenomena (e.g., respiration and cardiac pulse), then an important aspect of quality of data is periodicity of signal. Pulse waveforms and respiratory waveforms have periodically recurring peaks. The more periodic the waveform, the higher the magnitude of the highest magnitude peak in the frequency domain. Put differently, the more periodic the waveform, the higher the magnitude of the frequency component that has the highest magnitude. In some cases, if a first and second estimate of a physiological parameter are derived from a first and second waveform, respectively, then the two estimates may be aggregated by taking a weighted average of the two estimates, where (i) the weight applied to the first estimate equals the magnitude of the highest magnitude component of the FFT of the first waveform, and (ii) the weight applied to the second estimate equals the magnitude of the highest magnitude component of the FFT of the second waveform.

In some cases, large motion artifacts (e.g., caused by person typing, by a person walking or running, or by rapid acceleration of a car that a user is driving) are an important aspect of quality of data. For example, if rapid linear acceleration in the time domain is detected, then during the acceleration, data from accelerometers (which are affected by the acceleration) may be disregarded or weighted by a lower weight and data from a gyroscope (which is not affected by the acceleration) may be given a higher weight.

In some embodiments of this invention, the amount of motion measured by a specific stream of sensor data is determined. For each specific stream of sensor data, this determination involves computing the first derivative of the sensor data, aggregating the different components (L2 norm), and, computing the standard deviation. Too much or too little standard deviation tends to negatively impact the quality of the readings.

User-Inputted Data: In some cases, certain trigger factors are inputted by a user. For example, in some cases, one or more I/O devices (e.g., 241-246) accept input from a user. The input specifies the identity, gender, weight or age of the user. Alternatively, a computer (e.g., 236) analyzes sensor readings to biometrically identify a user, and then accesses (or downloads from the Internet) data that is stored in electronic memory and that specifies the gender, weight or age of the user. The data stored in memory may have been inputted earlier by the user, or may have been determined by a computer by feature analysis.

Activity: In some cases, the type of activity in which a user is engaged is a trigger factor that affects weighting. For example, in some cases: (a) the sensor system includes multiple sensor modules; (b) one of the sensor modules is housed in a mobile computing device (MCD), such as a smartphone; (c) the MCD detects a type of activity that the user is engaged in, such as listening to music on the MCD, browsing the Internet via the MCD, or reading an e-book on an MCD screen. In some cases, a certain type of activity (such as typing, walking, or running) produce large motion artifacts that are determined by sensors (e.g., accelerometers or gyroscopes) in sensor modules of the sensor system.

Feature Analysis: In some cases, a computer analyses features in pulse or respiratory waveforms in order to determine trigger factors.

In some implementations, one or more trigger factors are determined by feature analysis of pulse or respiratory waveforms.

The following five paragraphs describe a prototype of this invention, in which feature analysis from pulse waveforms is performed. This prototype is a non-limiting example of how feature analysis may be performed.

In this prototype, a head-mounted sensor module and wrist-worn sensor module each include a 3-axis accelerometer and a 3-axis gyroscope. While the accelerometer captures linear accelerations (meters/second), the gyroscope captures the rate of rotation (radians/second) of the device. The average sampling rates are 50 Hz and 100 Hz for the head-mounted and wrist-worn modules, respectively. However, the streams of data are interpolated to a constant sampling rate of 256 Hz.

In this prototype, for purposes of training the sensor system, six separate one-minute recordings are taken per individual user, for twelve individual users. The six recordings per user are: each user is recorded in three postures (standing, sitting and lying down) and for each posture, before and after exercising. In order to create several sample readings for each of the conditions, the data is split into non-overlapping segments of 10 seconds each, yielding 432 segments equally balanced in terms of person and body posture. For each of these segments, several processing steps are employed to amplify BCG motions and to extract representative features. Each sensor modality (accelerometer and gyroscope) is processed separately.

In this prototype, a pulse waveform is recovered from subtle body movements as follows: First, normalize each of the 10-second sensor components (e.g., each axis of the 3 axis of the accelerometer) to have zero mean and unit variance. Next, subtract an averaging filter (window of 35 samples) to detrend the data and to remove relatively slow motions such as respiration and stabilizing body motions. Next, use a Butterworth band-pass filter (cut-off frequencies of 4-11 Hz) to recover the BCG waveform from each component. As not all the components carry relevant cardiac information, select the most periodic component of each sensor modality (accelerometer and gyroscope) by choosing the signal with highest amplitude response in the frequency domain.

In this prototype, features are extracted that may be used to characterize each of the measurements. The feature extraction includes segmenting the parts of the signal associated with different heartbeats, computing the average beat response, and extracting representative features from it. More specifically, the feature extraction includes the following steps: First, locate potential heart beat responses with the findpeaks MATLAB® function (with MIN_PEAK_DISTANCE equal to the length of a heartbeat when the heart rate is 150). (Reason: each heartbeat is characterized by a larger motion peak surrounded by smaller ones.) Then segment the signals by taking 300 milliseconds before and 500 milliseconds after each of the previous peaks. Next, average the different segments resulting in a specific BCG beat response. Next, extract the following features: 1) raw amplitude values, 2) histogram capturing the distribution of values (200 bins), and 3) shape features. For the shape features, extract the angles and distances between five descriptive points that are known to vary due to different trigger factors. Detect the descriptive points by splitting the signal into halves and using the findpeaks function to obtain the maximum and minimum values of each subsegment.

In this prototype, features are classified by a computer executing a linear Support Vector Machine algorithm with probability estimates, which allow for multiple class labels. Specifically, the algorithm uses the libSVM library which offers an efficient MATLAB® implementation. The misclassification cost is optimized with a 10 fold-cross validation approach on the training set. In other words, the training data is divided into 10 groups. Then, train on nine and test on the tenth and repeat the process for each of the groups to find which value yields the highest average classification accuracy. The considered values for misclassification cost are: log₂ C, for C={−10, −9, −8, . . . 10}. In order to give the same relevance to each feature type, all the features are standardized to have zero mean and unit variance before training. Moreover, the dimensionality of the feature vector is reduced with Principal Component Analysis (preserving 95% of the energy), resulting in fewer than 100 components per condition.

This invention is not limited to the prototype described above. Feature analysis may be implemented in many other ways, in illustrative implementations of this invention.

FIG. 13 shows steps in a method of determining trigger factors by feature analysis of waveforms, in an illustrative implementation of this invention. The method shown in FIG. 13 includes the following steps: Sensors (including one or more gyroscopes, accelerometers and PPG sensors) take measurements of subtle body motions caused by a user's heartbeat and respiration and of blood volume pulse (Step 1301). A computer and signal processor process sensor data indicative of the measurements (Step 1303). A computer calculates one or more BCG pulse waveforms and BCG respiratory waveforms, based on processed sensor data from the gyroscope(s) and accelerometer(s). A computer calculates one or more PPG pulse waveforms and PPG respiratory waveforms, based on processed sensor data from the PPG sensor(s) (Step 1305). A computer performs features analysis, based at least on the waveforms (Step 1307). A computer determines, based at least on the features analysis, one or more of the following trigger factors: posture (e.g., whether a user is standing, sitting up, lying down), identity of user, gender of user, physical weight of user, or age of user. (Step 1309).

### Field of Endeavor and Problem Faced by the Inventors

A field of endeavor of this invention is dynamic weighting of data from a set of sensors, such that weighting changes over time and depends on at least (i) type of sensor and (ii) sensor position relative to a user's body.

The inventors were faced by a problem: The problem is how to dynamically weight data from a set of sensors, such that weighting changes over time and depends on at least (i) type of sensor and (ii) sensor position relative to a user's body.

### Computers

In exemplary implementations of this invention, one or more electronic computers (e.g. 206, 216, 226, 236) are programmed and specially adapted: (1) to control the operation of, or interface with, hardware components of a sensor system, including any sensor module, accelerometer, gyroscope, PPG sensor, camera, EDA sensor, thermometer, or wireless transceiver unit; (2) to process sensor measurements to compute a cardiac pulse waveform or a respiratory waveform; (3) to extract physiological parameters from the pulse and respiratory waveforms, including heart rate, breathing rate, and heart rate variability; (4) to dynamically adjust the weight given to sensor data gathered by different sensors or the weight given to information that is calculated from such sensor data, such as pulse waveforms, respiratory waveforms, or estimated HR, HRV and BR; (5) to calculate the weights used in the dynamical adjustment, based on one or more trigger factors including position of sensor, posture of user, identity of user, gender of user, weight of user, age of user, activity of user (e.g., watching a movie, listening to music, or browsing), availability of data, quality of data, and what the weighted average is computing; (6) to determine one or more trigger factors based at least in part on (i) user input, (ii) activities of user detected by a smartphone or other mobile computing device, or (iii) feature analysis of a cardiac or respiratory waveform; (7) to perform any other calculation, computation, program, algorithm, computer function or computer task described or implied above; (8) to receive signals indicative of human input; (9) to output signals for controlling transducers for outputting information in human perceivable format; and (10) to process data, to perform computations, to execute any algorithm or software, and to control the read or write of data to and from memory devices. The one or more computers may be in any position or positions within or outside of the sensor system. For example, in some cases (a) at least one computer is housed in or together with other components of the sensor system, such as a sensor module, and (b) at least one computer is remote from other components of the sensor system. The one or more computers are connected to each other or to other components in the sensor system either: (a) wirelessly, (b) by wired connection, (c) by fiber-optic link, or (d) by a combination of wired, wireless or fiber optic links.

In exemplary implementations, one or more computers are programmed to perform any and all calculations, computations, programs, algorithms, computer functions and computer tasks described or implied above. For example, in some cases: (a) a machine-accessible medium has instructions encoded thereon that specify steps in a software program; and (b) the computer accesses the instructions encoded on the machine-accessible medium, in order to determine steps to execute in the program. In exemplary implementations, the machine-accessible medium comprises a tangible non-transitory medium. In some cases, the machine-accessible medium comprises (a) a memory unit or (b) an auxiliary memory storage device. For example, in some cases, a control unit in a computer fetches the instructions from memory.

In illustrative implementations, one or more computers execute programs according to instructions encoded in one or more tangible, non-transitory, computer-readable media. For example, in some cases, these instructions comprise instructions for a computer to perform any calculation, computation, program, algorithm, computer function or computer task described or implied above. For example, in some cases, instructions encoded in a tangible, non-transitory, computer-accessible medium comprise instructions for a computer to: 1) to control the operation of, or interface with, hardware components of a sensor system, including any sensor module, accelerometer, gyroscope, PPG sensor, camera, EDA sensor, thermometer, or wireless transceiver unit; (2) to process sensor measurements to compute a cardiac pulse waveform or a respiratory waveform; (3) to extract physiological parameters from the pulse and respiratory waveforms, including heart rate, breathing rate, and heart rate variability; (4) to dynamically adjust the weight given to sensor data gathered by different sensors or the weight given to information that is calculated from such sensor data, such as pulse waveforms, respiratory waveforms, or estimated HR, HRV and BR; (5) to calculate the weights used in the dynamical adjustment, based on one or more trigger factors including posture of user, identity of user, gender of user, weight of user, age of user, activity of user (e.g., watching a movie, listening to music, or browsing), availability of data, quality of data, and what the weighted average is computing; (6) to determine one or more trigger factors based at least in part on (i) user input, (ii) activities of user detected by a smartphone or other mobile computing device, or (iii) feature analysis of a cardiac or respiratory waveform computed from sensor readings; (7) to perform any other calculation, computation, program, algorithm, computer function or computer task described or implied above; (8) to receive signals indicative of human input; (9) to output signals for controlling transducers for outputting information in human perceivable format; and (10) to process data, to perform computations, to execute any algorithm or software, and to control the read or write of data to and from memory devices.

### Network Communication

In illustrative implementations of this invention, an electronic device (e.g., any of devices 201-204, 206-208, 211-214, 216-219, 221-224, 226-228, 236) is configured for wireless or wired communication with other electronic devices in a network.

For example, in some cases, a computer 236 and sensor modules 201, 211, 221 each include a wireless transceiver unit for wireless communication with other electronic devices in a network. Each wireless transceiver unit (e.g., 205, 215, 225, 235) includes (a) one or more antennas, (b) one or more wireless transceivers, transmitters or receivers, and (c) signal processing circuitry. The wireless transceiver unit receives and transmits data in accordance with one or more wireless standards.

In some cases, one or more of the following hardware components are used for network communication: a computer bus, a computer port, network connection, network interface device, host adapter, wireless transceiver unit, wireless card, signal processor, modem, router, computer port, cables or wiring.

In some cases, one or more computers (e.g., 206, 216, 226, 236) are programmed for communication over a network. For example, in some cases, one or more computers are programmed for network communication: (a) in accordance with the Internet Protocol Suite, or (b) in accordance with any other industry standard for communication, including any USB standard, ethernet standard (e.g., IEEE 802.3), token ring standard (e.g., IEEE 802.5), wireless standard (including IEEE 802.11 (wi-fi), IEEE 802.15 (bluetooth/zigbee), IEEE 802.16, IEEE 802.20 and including any mobile phone standard, including GSM (global system for mobile communications), UMTS (universal mobile telecommunication system), CDMA (code division multiple access, including IS-95, IS-2000, and WCDMA), or LTS (long term evolution)), or other IEEE communication standard.

### DEFINITIONS

The terms “a” and “an”, when modifying a noun, do not imply that only one of the noun exists.

To say that X is “adjacent” to Y means that X physically touches Y.

The terms “ballistocardiographic” and “BCG” are defined above.

To compute “based on” specified data means to perform a computation that takes the specified data as an input.

Here are some non-limiting examples of a “camera”: (a) a digital camera; (b) a video camera; (c) a light sensor, (d) a set or array of light sensors; (e) an imaging system; (f) a light field camera or plenoptic camera; (g) a time-of-flight camera; or (h) an optical instrument that records images. A camera includes any computers or circuits that process data captured by the camera.

The term “comprise” (and grammatical variations thereof) shall be construed as if followed by “without limitation”. If A comprises B, then A includes B and may include other things.

The term “computer” includes any computational device that performs logical and arithmetic operations. For example, in some cases, a “computer” comprises an electronic computational device, such as an integrated circuit, a microprocessor, a mobile computing device, a laptop computer, a tablet computer, a personal computer, or a mainframe computer. In some cases, a “computer” comprises: (a) a central processing unit, (b) an ALU (arithmetic logic unit), (c) a memory unit, and (d) a control unit that controls actions of other components of the computer so that encoded steps of a program are executed in a sequence. In some cases, a “computer” also includes peripheral units including an auxiliary memory storage device (e.g., a disk drive or flash memory), or includes signal processing circuitry. However, a human is not a “computer”, as that term is used herein.

“Defined Term” means a term or phrase that is set forth in quotation marks in this Definitions section.

To say that Y “depends at least in part on” X means that Y depends at least in part on (i) X, (ii) an estimate of X, (iii) a probability regarding X, or (iv) a degree of membership in a fuzzy set X. For example, to say that Y “depends at least in part on” a person's weight means that Y depends at least in part on (i) the person's actual weight or weight range, (ii) an estimate of the person's weight or weight range, (iii) a probability regarding the person's weight or weight range, or (iv) a degree of membership in a fuzzy set regarding weight. To say that Y “depends at least in part on” a person's age means that Y depends at least in part on (i) the person's actual age or age range, (ii) an estimate of the person's age or age range, (iii) a probability regarding the person's age or age range, or (iv) a degree of membership in a fuzzy set regarding age.

For an event to occur “during” a time period, it is not necessary that the event occur throughout the entire time period. For example, an event that occurs during only a portion of a given time period occurs “during” the given time period.

The term “e.g.” means for example.

The fact that an “example” or multiple examples of something are given does not imply that they are the only instances of that thing. An example (or a group of examples) is merely a non-exhaustive and non-limiting illustration.

Unless the context clearly indicates otherwise: (1) a phrase that includes “a first” thing and “a second” thing does not imply an order of the two things (or that there are only two of the things); and (2) such a phrase is simply a way of identifying the two things, respectively, so that they each may be referred to later with specificity (e.g., by referring to “the first” thing and “the second” thing later). For example, unless the context clearly indicates otherwise, if an equation has a first term and a second term, then the equation may (or may not) have more than two terms, and the first term may occur before or after the second term in the equation. A phrase that includes a “third” thing, a “fourth” thing and so on shall be construed in like manner.

“For instance” means for example.

To say that data is “from” a sensor means that the data represents measurements taken by the sensor or represents information calculated from the measurements.

“Herein” means in this document, including text, specification, claims, abstract, and drawings.

As used herein: (1) “implementation” means an implementation of this invention; (2) “embodiment” means an embodiment of this invention; (3) “case” means an implementation of this invention; and (4) “use scenario” means a use scenario of this invention.

The term “include” (and grammatical variations thereof) shall be construed as if followed by “without limitation”.

“I/O device” means an input/output device. Non-limiting examples of an I/O device include any device for (a) receiving input from a human user, (b) providing output to a human user, or (c) both. Non-limiting examples of an I/O device also include a touch screen, other electronic display screen, keyboard, mouse, microphone, handheld electronic game controller, digital stylus, display screen, speaker, or projector for projecting a visual display.

The term “mobile computing device” or “MCD” means a device that includes a computer, a camera, a display screen and a wireless transceiver. Non-limiting examples of an MCD include a smartphone, cell phone, mobile phone, tablet computer, laptop computer and notebook computer.

The term “or” is inclusive, not exclusive. For example A or B is true if A is true, or B is true, or both A or B are true. Also, for example, a calculation of A or B means a calculation of A, or a calculation of B, or a calculation of A and B.

As used herein, “parameter” means a variable. For example: (a) if y=f(x), then both x and y are parameters; and (b) if z=f(x(t), y(t)), then t, x, y and z are parameters. For example, in some cases a parameter is a physiological variable, such as heart rate, heart rate variability or respiration rate.

“Physiological gender” of a person means the sex (male or female) of person indicated by the reproductive organs of the person.

As used herein, the term “set” does not include a group with no elements. Mentioning a first set and a second set does not, in and of itself, create any implication regarding whether or not the first and second sets overlap (that is, intersect).

“Some” means one or more.

As used herein, a “subset” of a set consists of less than all of the elements of the set.

“Substantially” means at least ten percent. For example: (a) 112 is substantially larger than 100; and (b) 108 is not substantially larger than 100.

The term “such as” means for example.

As used herein, “trigger factor” means a factor that affects a weight that is given to data.

To say that a machine-readable medium is “transitory” means that the medium is a transitory signal, such as an electromagnetic wave.

To say that two sensors are “a single type of motion sensor” means that the two sensors are both accelerometers or are both gyroscopes.

Except to the extent that the context clearly requires otherwise, if steps in a method are described herein, then the method includes variations in which: (1) steps in the method occur in any order or sequence, including any order or sequence different than that described; (2) any step or steps in the method occurs more than once; (3) different steps, out of the steps in the method, occur a different number of times during the method, (4) any combination of steps in the method is done in parallel or serially; (5) any step or steps in the method is performed iteratively; (6) a given step in the method is applied to the same thing each time that the given step occurs or is applied to different things each time that the given step occurs; or (7) the method includes other steps, in addition to the steps described.

This Definitions section shall, in all cases, control over and override any other definition of the Defined Terms. For example, the definitions of Defined Terms set forth in this Definitions section override common usage or any external dictionary. If a given term is explicitly or implicitly defined in this document, then that definition shall be controlling, and shall override any definition of the given term arising from any source (e.g., a dictionary or common usage) that is external to this document. If this document provides clarification regarding the meaning of a particular term, then that clarification shall, to the extent applicable, override any definition of the given term arising from any source (e.g., a dictionary or common usage) that is external to this document. To the extent that any term or phrase is defined or clarified herein, such definition or clarification applies to any grammatical variation of such term or phrase, taking into account the difference in grammatical form. For example, the grammatical variations include noun, verb, participle, adjective, and possessive forms, and different declensions, and different tenses. In each case described in this paragraph, the Applicant or Applicants are acting as his, her, its or their own lexicographer.

### Variations

This invention may be implemented in many different ways. Here are some non-limiting examples:

In one aspect, this invention is a sensor system that comprises a set of sensors for measuring motion of a user's body, which set of sensors includes one or more gyroscopes and one or more accelerometers, wherein the sensor system is configured: (a) to make estimations of one or more physiological parameters of a user, based on data from the set of sensors, (b) to assign different weights to data from different sensors when making the estimations, such that (i) for at least one estimation, a weight assigned to data from at least one gyroscope is different than a weight assigned to data from at least one accelerometer; (ii) for at least one estimation, a weight assigned to data from a first sensor located in a first region relative to the user's body is different than a weight assigned to data from a second sensor located in a second region relative to the user's body, which first and second regions do not intersect, the first and second sensors being a single type of motion sensor; and (iii) a weight assigned to data from at least one sensor changes from at least one estimation to another estimation. In some cases, a weight assigned to a sensor depends at least in part on whether a user is standing, sitting or lying down. In some cases, a weight assigned to data from a specific sensor depends at least in part on periodicity of a signal measured by the specific sensor. In some cases, a weight assigned to data from a given sensor depends at least in part on magnitude of the highest magnitude frequency component of the data from the given sensor. In some cases, a weight assigned to data from a sensor depends at least in part on identity of the user. In some cases, a weight assigned to data from a sensor depends at least in part on physiological gender of the user. In some cases, a weight assigned to data from a sensor depends at least in part on age of the user. In some cases, a specific weight assigned to data from a sensor depends at least in part on what is being calculated, in a calculation that involves a multiplication of a term by the given weight. In some cases, a weight assigned to data from a particular sensor depends at least in part on magnitude of linear acceleration measured by the particular sensor. In some cases, the first and second regions are selected from a set of regions that includes (a) a region adjacent to the user's head, and (b) a region that is adjacent to the user's wrist and that does not intersect the region adjacent to the user's head. In some cases, the one or more physiological parameters include cardiac pulse rate. In some cases, the one or more physiological parameters include respiratory rate. In some cases, the one or more physiological parameters include heart rate variability. In some cases, the sensor system is configured: (a) to make a biometric identification of the identity of the user, based at least in part on measurements taken by the one or more accelerometers and one or more gyroscopes; and (b) to assign different weights to data from different sensors, when making the biometric identification. In some cases, the sensor system is configured to assign different weights to data from different sensors, such that, when making the biometric identification, a weight assigned to data from at least one gyroscope is different than a weight assigned to data from at least one accelerometer. In some cases, the sensor system is configured to assign different weights to data from different sensors, such that, when making the biometric identification, a weight assigned to data from a sensor (Sensor A) located in a first region relative to the user's body is different than a weight assigned to data from a sensor (Sensor B) located in a second region relative to the user's body, which first and second regions do not intersect, Sensor A and Sensor B being a single type of motion sensor. In some cases: (a) the sensor system includes one or more optical sensors for measuring light that reflects from or is transmitted through skin; and (b) the sensor system is configured to assign different weights to data from different sensors when making the estimations, such that for at least one estimation, a weight assigned to data from at least one optical sensor is different than a weight assigned to data from at least one accelerometer or from at least one gyroscope. In some cases, at least one optical sensor is a photoplethysmographic sensor. In some cases, at least one optical sensor is a camera that measures motion of a scene relative to the user. Each of the cases described above in this paragraph is an example of the sensor system described in the first sentence of this paragraph, and is also an example of an embodiment of this invention that may be combined with other embodiments of this invention.

In another aspect, this invention is a method comprising, in combination: (a) a set of sensors measuring motion of a user's body, which set of sensors includes one or more gyroscopes and one or more accelerometers; and (b) one or more computers making estimations of one or more physiological parameters of a user, based on data from the set of sensors, such that (i) for at least one estimation, a weight assigned to data from at least one gyroscope is different than a weight assigned to data from at least one accelerometer; and (ii) for at least one estimation, a weight assigned to data from a first sensor located in a first region relative to the user's body is different than a weight assigned to data from a second sensor located in a second region relative to the user's body, which first and second regions do not intersect, the first and second sensors being a single type of motion sensor; and (iii) a weight assigned to data from at least one sensor changes over time. The method described in the first sentence of this paragraph is an example of an embodiment of this invention that may be combined with other embodiments of this invention.

The above description (including without limitation any attached drawings and figures) describes illustrative implementations of the invention. However, the invention may be implemented in other ways. The methods and apparatus which are described above are merely illustrative applications of the principles of the invention. Other arrangements, methods, modifications, and substitutions by one of ordinary skill in the art are therefore also within the scope of the present invention. Numerous modifications may be made by those skilled in the art without departing from the scope of the invention. Also, this invention includes without limitation each combination and permutation of one or more of the abovementioned implementations, embodiments and features.

What is claimed is:

1. A sensor system that comprises a set of sensors for measuring motion of a user's body, which set of sensors includes one or more gyroscopes and one or more accelerometers, wherein the sensor system is configured:
(a) to make estimations of one or more physiological parameters of a user, based on data from the set of sensors,
(b) to assign different weights to data from different sensors when making the estimations, such that
(i) for at least one estimation, a weight assigned to data from at least one gyroscope is different than a weight assigned to data from at least one accelerometer;
(ii) for at least one estimation, a weight assigned to data from a first sensor located in a first region relative to the user's body is different than a weight assigned to data from a second sensor located in a second region relative to the user's body, which first and second regions do not intersect, the first and second sensors being a single type of motion sensor; and
(iii) a weight assigned to data from at least one sensor changes from at least one estimation to another estimation.

2. The sensor system of claim 1, wherein a weight assigned to a sensor depends at least in part on whether a user is standing, sitting or lying down.

3. The sensor system of claim 1, wherein a weight assigned to data from a specific sensor depends at least in part on periodicity of a signal measured by the specific sensor.

4. The sensor system of claim 1, wherein a weight assigned to data from a given sensor depends at least in part on magnitude of the highest magnitude frequency component of the data from the given sensor.

5. The sensor system of claim 1, wherein a weight assigned to data from a sensor depends at least in part on identity of the user.

6. The sensor system of claim 1, wherein a weight assigned to data from a sensor depends at least in part on physiological gender of the user.

7. The sensor system of claim 1, wherein a weight assigned to data from a sensor depends at least in part on age of the user.

8. The sensor system of claim 1, wherein a specific weight assigned to data from a sensor depends at least in part on what is being calculated, in a calculation that involves a multiplication of a term by the given weight.

9. The sensor system of claim 1, wherein a weight assigned to data from a particular sensor depends at least in part on magnitude of linear acceleration measured by the particular sensor.

10. The sensor system of claim 1, wherein the first and second regions are selected from a set of regions that includes (a) a region adjacent to the user's head, and (b) a region that is adjacent to the user's wrist and that does not intersect the region adjacent to the user's head.

11. The sensor system of claim 1, wherein the one or more physiological parameters include cardiac pulse rate.

12. The sensor system of claim 1, wherein the one or more physiological parameters include respiratory rate.

13. The sensor system of claim 1, wherein the one or more physiological parameters include heart rate variability.

14. The sensor system of claim 1, wherein the sensor system is configured:
(a) to make a biometric identification of the identity of the user, based at least in part on measurements taken by the one or more accelerometers and one or more gyroscopes; and
(b) to assign different weights to data from different sensors, when making the biometric identification.

15. The sensor system of claim 14, wherein the sensor system is configured to assign different weights to data from different sensors, such that, when making the biometric identification, a weight assigned to data from at least one gyroscope is different than a weight assigned to data from at least one accelerometer.

16. The sensor system of claim 14, wherein the sensor system is configured to assign different weights to data from different sensors, such that, when making the biometric identification, a weight assigned to data from a sensor (Sensor A) located in a first region relative to the user's body is different than a weight assigned to data from a sensor (Sensor B) located in a second region relative to the user's body, which first and second regions do not intersect, Sensor A and Sensor B being a single type of motion sensor.

17. The sensor system of claim 1, wherein:
(a) the sensor system includes one or more optical sensors for measuring light that reflects from or is transmitted through skin; and
(b) the sensor system is configured to assign different weights to data from different sensors when making the estimations, such that for at least one estimation, a weight assigned to data from at least one optical sensor is different than a weight assigned to data from at least one accelerometer or from at least one gyroscope.

18. The sensor system of claim 17, wherein at least one optical sensor is a photoplethysmographic sensor.

19. The sensor system of claim 17, wherein at least one optical sensor is a camera that measures motion of a scene relative to the user.

20. A method comprising, in combination:
(a) a set of sensors measuring motion of a user's body, which set of sensors includes one or more gyroscopes and one or more accelerometers; and
(b) one or more computers making estimations of one or more physiological parameters of a user, based on data from the set of sensors, such that
(i) for at least one estimation, a weight assigned to data from at least one gyroscope is different than a weight assigned to data from at least one accelerometer; and
(ii) for at least one estimation, a weight assigned to data from a first sensor located in a first region relative to the user's body is different than a weight assigned to data from a second sensor located in a second region relative to the user's body, which first and second regions do not intersect, the first and second sensors being a single type of motion sensor; and
(iii) a weight assigned to data from at least one sensor changes over time.
